(19) United States
(12) Patent Application Publication
(10) Pub. No.: US 2023/0357753 A1
(43) Pub. Date: Nov. 9, 2023

(54) Methods and Systems for Determining a Minimum Number of Cell Line Clones Necessary to Produce a Product Having a Set of Target Product Attributes

(71) Applicant: AMGEN INC., Thousand Oaks, CA (US)

(72) Inventors: Huong Thi Ngoc Le, Thousand Oaks, CA (US); Jasmine Tat, Thousand Oaks, CA (US); Ewelina Zasadzinska, Thousand Oaks, CA (US)

(21) Appl. No.: 18/026,264

(22) PCT Filed: Sep. 9, 2021
(86) PCT No.: PCT/US2021/049562

§ 371 Date: Mar. 14, 2023

Related U.S. Application Data
(60) Provisional application No. 63/082,682, filed on Sep. 24, 2020.

(51) Int. Cl. C12N 15/10; G16B 25/10; C12M 1/36

(52) U.S. Cl. CPC C12N 15/1034; C12M 41/48; G16B 25/10

(57) ABSTRACT

Methods and systems for determining a minimum number of cell line clones necessary to produce a product having a set of target product attributes are disclosed. An example method includes generating at least one cell line capable of expressing a polypeptide; measuring, using one or more analytical instruments, a plurality of measured product attribute values of a plurality of clones of a candidate cell line; receiving inputs, via a user interface, representing a set of target product attribute values for a product; projecting, by one or more processors based upon the plurality of measured values, a minimum number of subject clones of the product using the candidate cell line necessary to produce a subset of the subject clones having product attributes that satisfy one or more conditions associated with the set of target values; and generating the projected minimum number of subject clones of the product using the candidate cell line.

### CROSS-REFERENCE TO RELATED APPLICATION(S)

This application claims the priority benefit of U.S. Provisional Pat. Application No. 63/082,682, filed Sep. 24, 2020, which is hereby incorporated by reference in its entirety.

### FIELD OF DISCLOSURE

The present application relates generally to cell line cloning and, more specifically, to methods and systems for determining a minimum number of cell line clones necessary to produce a product having a set of target product attributes.

### BACKGROUND

In the biopharmaceutical industry, large, complex molecules (e.g., proteins) known as biologics are derived from living systems. The general workflow for the development of a biologic begins with research and development. In this initial phase, a disease, or indication that represents an important unmet medical need is targeted. Researchers determine the potential drug candidates based upon a proper target product profile, which govern aspects such as safety, efficacy, and route of administration, for example. Ultimately, through a combination of in vitro research and computational models, a specific molecule is chosen as the top drug candidate for the specific disease and target population. After the top candidate is selected, the blueprint for the molecule is formalized into a gene, and the gene of interest is inserted into an expression vector. The expression vector is then inserted into a host cell, in a process known as transfection. The host cell can incorporate the gene of interest into its own production mechanisms upon successful transfection, eventually gaining the ability to produce the desired pharmaceutical product.

Because each cell has unique characteristics, the product produced by each cell varies slightly, e.g., in terms of productivity (e.g., titer) and product quality. In general, it is more desirable to produce drugs with consistently high titers and consistently high quality, for reasons of safety and economy. High concentrations, or titers, of a product help to reduce the manufacturing footprint needed to generate desired production volumes, and therefore save both capital and operating expenses. High product quality ensures that the drug is safe, efficacious, and usable, which also reduces costs. In the context of cell line development, product quality attributes are evaluated through assays conducted on the product of interest. These assays often include chromatographic analysis, which is used to determine attributes such as degree of glycosylation and other factors such as the proportion of unusable proteins due to truncations (clippings) or clumping (aggregates).

Based upon criteria relating to productivity and product quality, the “best” cell line or clone is selected in a process known as “cell line selection,” “clone selection,” or “clone screening.” The selected cell line/clone is used for the master cell bank, which serves as the homogeneous starting point for all future manufacturing (e.g., clinical and commercial).

Ensuring a consistent product batch helps promote a more uniform and predictable pharmacokinetic and pharmacodynamic response in patients. If a “pool” of heterogeneous cells obtained after transfection is used to generate the product of interest, however, there may be many variants of the product generated. This is because during transfection, the gene of interest is integrated into candidate host cells in variable ways. For example, there may be differences in copy number (i.e., the number of integrated copies of the gene of interest), the integration site (i.e., locations in the host genome where the gene of interest integrates to) and other differentiating factors between the unique footprints of different cells. The manufacturing of the product of interest may also vary due to slight differences in the internal machinery of each individual cell, including the nature of post-translational modifications. These variations are undesirable, especially considering the need to ultimately control for and ensure a safe and measured response in the patient. Thus, it is typically required that the master cell bank cell line be “clonally derived,” i.e., that the master cell bank only contain cells derived from a common, single cell ancestor. This theoretically helps ensure a large degree of homogeneity in the drug produced, despite slight, inevitable differences due to natural genetic variation through random mutation as cells divide. Therefore, the clone screening process is important in delivering not only a productive, high quality starting material, but also a singular cell line that complies with the “clonally derived” requirement from regulatory agencies.

FIG. 1 depicts a typical clone screening process 100. A first stage 110 depicts the traditional microtiter plate-based method of clone generation and growth, which starts with 1 cell per microtiter plate well and may take two to three weeks. Hundreds of pooled, heterogeneous cells are sorted into single-cell cultures through processes such as fluorescence-activated cell sorting (FACS) or limiting dilution. After being allowed to recover to healthy and stable populations, these clonally-derived cells are analyzed, and select populations are transferred to a second stage 120. At the second stage 120, clonal cells in small vessels, such as spin tubes or deep well plates are cultured in a “small-scale production”. In this small-scale process, boluses of nutrients are added periodically, and different measurements of cell growth and viability are obtained. Typically, hundreds or even thousands of these small-scale cultures are run in parallel. At the end of the culture, the supernatants or medium are harvested for assays and analyses of the secreted products.

By analyzing the growth and productivity characteristics of the clones in the small-scale cultures, at the second stage 120, the “top” or “best” clones (e.g., the top four) are selected for scaled-up cultures that are run at a third stage 130. The scaled-up (or “large-scale”) process is useful because, relative to the small-scale cultures at the second stage 120, it better represents the process that will ultimately be used in clinical and commercial manufacturing. A higher number of measured variables, such as daily and continuous process conditions and metabolite concentrations, are typically measured during the bioreactor process to enable tighter control and monitoring.

After the scaled-up process at the third stage 130, the product is collected and analyzed. Ultimately, at a fourth stage 140, the scaled-up run that yielded the highest titer and exhibited the best product quality attributes (PQA) is typically chosen as the “best,” or “winning,” clone. Finally, at a fifth stage 150, the winning clone is used to generate the master cell bank for future clinical and commercial manufacturing use.

Conventional clone screening processes of the sort described above are extremely costly and resource-intensive, typically taking several months and requiring hundreds or thousands of assays and cell cultures. As the pace of biotechnology quickens, there is an increasing need for reducing the number of clones that need to be generated and screened.

### SUMMARY

Embodiments described herein relate to methods and systems for determining the minimum number of cell line clones necessary to produce or result in a product having a set of target product attributes. This minimum number of clones can be generated and assayed, rather than generating a predetermined number of clones which may be excessive or insufficient. By generating this minimum number of clones, products having a desired set of target product attributes can be generated with fewer resources, and/or without having to repeat the lengthy clone generation process when an insufficient number of clones are initially generated. Moreover, it can be identified, a priori, when a host cell line would likely not result in a product meeting a set of target product attributes. Furthermore, this minimum number of clones can be used at the planning stage to more accurately project the time and/or resources necessary to develop the desired products, thereby facilitating more predictable product development plans.

In an embodiment, a method includes generating at least one cell line capable of expressing a polypeptide, measuring, using one or more analytical instruments, a plurality of measured product attribute values of a plurality of clones of a candidate cell line; receiving inputs, via a user interface, representing a set of target product attribute values for a product; projecting, by one or more processors based upon the plurality of measured values, a minimum number of subject clones of the product using the candidate cell line necessary to produce a subset of the subject clones having product attributes that satisfy one or more conditions associated with the set of target values; and generating the projected minimum number of subject clones of the product using the candidate cell line.

In some aspects, projecting the minimum number of subject clones includes: computing a probability that one of the plurality of clones satisfies one or more conditions associated with the set of target values based upon a total number of the plurality of clones and a number of the plurality of clones having product attributes that satisfy the one or more conditions associated with set of target product attribute values; and projecting the minimum number of subject clones based upon the probability.

In some aspects, the probability is a first probability, and projecting the minimum subject clones includes: receiving, via a user interface, a confidence level value indicative of a second probability in which the subset of the subject clones results in at least a threshold number of clones having product attributes that satisfy the one or more conditions associated with the target values; and projecting the minimum number of subject clones as a function of the confidence level value, the first probability, and the threshold number of clones.

In some aspects, projecting the minimum number of subject clones includes solving for the minimum number N of subject clones given the threshold number k of clones satisfying the one or more conditions associated with the set of target product attribute values and the confidence level C is:

$$C = {\sum }_{j = 0}^{k - 1} \frac{N !}{j ! \left(N - j\right) !} {p}^{j} {\left(1 - p\right)}^{N - j} .$$

In some aspects, the threshold number of clones is one, the minimum number of subject clones (n) is determined as:

$$n = \frac{log \left(1 - C\right)}{log \left(1 - p\right)} ,$$

C is the confidence level value, and p is the first probability.

Some aspects further include measuring, using the one or more analytical instruments, a set of resultant product attribute values for each of the subject clones; and identifying one or more of the subject clones for additional testing based upon comparisons of the sets of measured resultant values and the set of target values.

### BRIEF DESCRIPTION OF THE DRAWINGS

The skilled artisan will understand that the figures, described herein, are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 depicts various stages of a typical clone screening process.

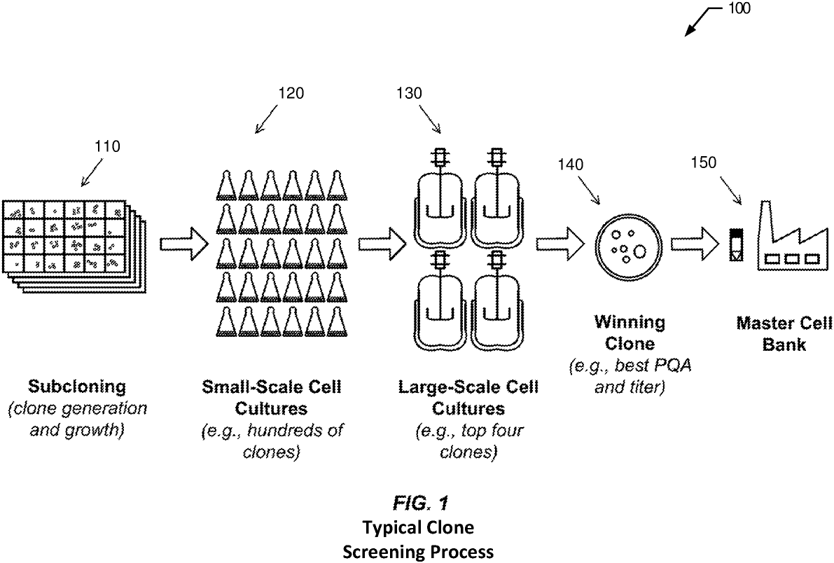

FIG. 2 is a block diagram of an example system to plan for and generate clones, in accordance with aspects of this disclosure.

FIG. 3 depicts an example dashboard, in accordance with aspects of this disclosure, that may be used to implement the example dashboard of FIG. 2.

FIG. 4 depicts example graphs showing sensitivities of the minimum number of clones needed to produce target products having target product attributes.

FIG. 5 is a table of example cell line cloning planning information.

FIG. 6 is a block diagram of an example computing system to implement the various user interfaces, methods, functions, etc., for determining a minimum number of cell line clones necessary to produce a product having a set of target product attributes, in accordance with the disclosed embodiments.

FIG. 7 is a flowchart representative of an example method, hardware logic or machine-readable instructions for implementing the example computing system of FIG. 6, in accordance with disclosed embodiments, to generate cell line clones.

### DETAILED DESCRIPTION

The various concepts introduced above and discussed in greater detail below may be implemented in any of numerous ways, and the described concepts are not limited to any particular manner of implementation. Examples of implementations are provided for illustrative purposes. Reference will now be made in detail to non-limiting examples, some of which are illustrated in the accompanying drawings.

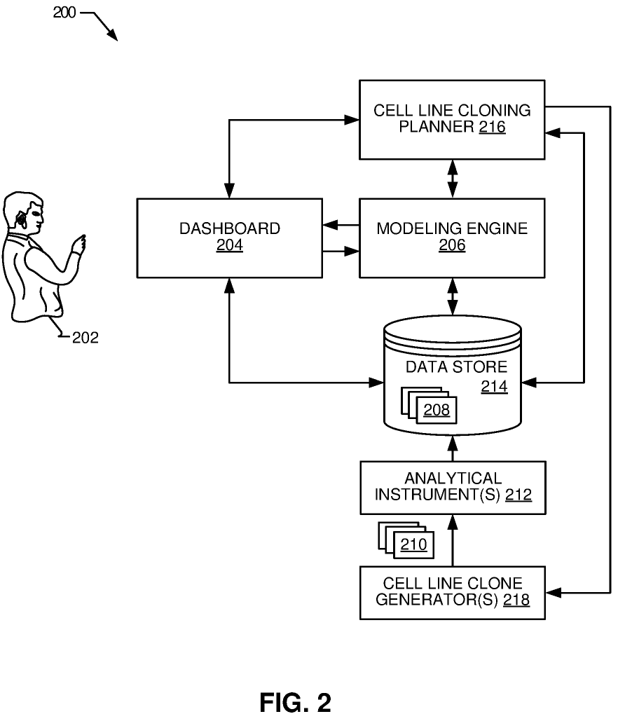

FIG. 2 is a block diagram of an example system 200, in accordance with aspects of this disclosure, that enables a user 202 to determine or project the minimum number of cell line clones statistically necessary to produce or result in a product having a set of target product attributes (also referred to herein as a “subset of the clones” or a “subclone”), and to generate those clones.

The system 200 includes a graphical user interface (GUI) in the form of a dashboard 204 that enables the user 202 to input one or more target product attribute values and review corresponding results. Example target product attribute values are values of titer (g/L), percentage high molecular weight (%HMW), percentage high mannose (%MAN), percentage afucosylation (%AFUC), percentage galactosylation (%GAL), percentage sialylation (%SIA), and doubling time (DT). The target product attribute values can be a single value for a product attribute, such as a titer value of at least 2.5 g/L. The target product attribute values can also be a range of values for a product attribute, such as a percentage afucosylation between 1.0% and 1.9%. Additionally, the target product attribute values can include target product attribute values for one product attribute (e.g., a titer value of at least 2.5 g/L), for two product attributes (e.g., a titer value of at least 2.5 g/L, and a percentage afucosylation between 1.0% and 1.9%) or any suitable number of product attributes. Example results include, but are not limited to, the minimum number of clones that should be generated based upon a set of target product attribute values, costs to generate the clones, sensitivity of the minimum number to product attribute values, etc. for different scenarios. Such results can be used to select which cell line(s) to clone, how many clones to generate, study impacts of changing target product attribute values, etc.

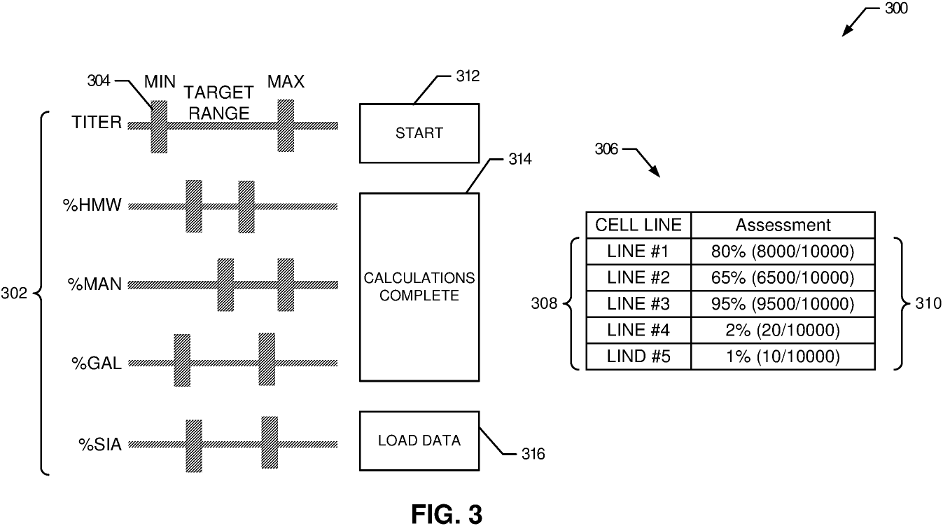

An example dashboard 300 that may be used to implement the dashboard 204 is shown in FIG. 3. In the dashboard 300, target product attribute values 302 can be set, specified, input, etc. by adjusting sliders (e.g., using a mouse or keyboard), one of which is designated by reference numeral 304. The sliders can be used to set a minimum, a maximum and a target range for respective product attributes. For example, the slider 304 sets a minimum titer for a current scenario being investigated. While sliders are used in the example of FIG. 3, other means of inputting target product attribute values may be used. For example, text input fields, boxes, drop down lists, import, etc.

Based upon the target product attribute values 302 set by the user 202 via the dashboard 204, 300, an example modeling engine 206 of FIG. 2 determines or projects the minimum number of cell line clones statistically necessary to produce or result in one or more products or subclones having product attributes that satisfy conditions associated with the set of target product attribute values (e.g., the target product attribute value is a titer value of at least 2 g/L, a subclone having a titer value greater than or equal to 2 g/L satisfies the condition associated with the titer value). The modeling engine 206 makes the determinations or projections based upon measured attributes 208 (e.g., titer, %HMW, %MAN, %AFUC, %GAL, %SIA, and DT) of prior, known cell line clones for one or more cell lines and/or one or more products. Such prior measurements may be captured for prior, known cell line clones 210 by one or more analytical instruments 212, and stored in a data store 214 using any number and/or type(s) of data structures.

The data store 214 may be implemented using any number and/or type(s) of volatile or non-volatile non-transitory computer- or machine-readable storage medium such as semiconductor memories, magnetically readable memories, optically readable memories, hard disk drive (HDD), an optical storage drive, a solid-state storage device, a solid-state drive (SSD), a read-only memory (ROM), a random-access memory (RAM), a compact disc (CD), a compact disc read-only memory (CD-ROM), a digital versatile disk (DVD), a Blu-ray disk, a redundant array of independent disks (RAID) system, a cache, a flash memory, or any other storage device or storage disk in which information may be stored for any duration (e.g., permanently, for an extended time period, for a brief instance, for temporarily buffering, for caching of the information, etc.). As used herein, the term non-transitory computer-readable medium is expressly defined to include any type of computer-readable storage device and/or storage disk and to exclude propagating signals and to exclude transmission media. As used herein, the term non-transitory machine-readable medium is expressly defined to include any type of machine-readable storage device and/or storage disk and to exclude propagating signals and to exclude transmission media.

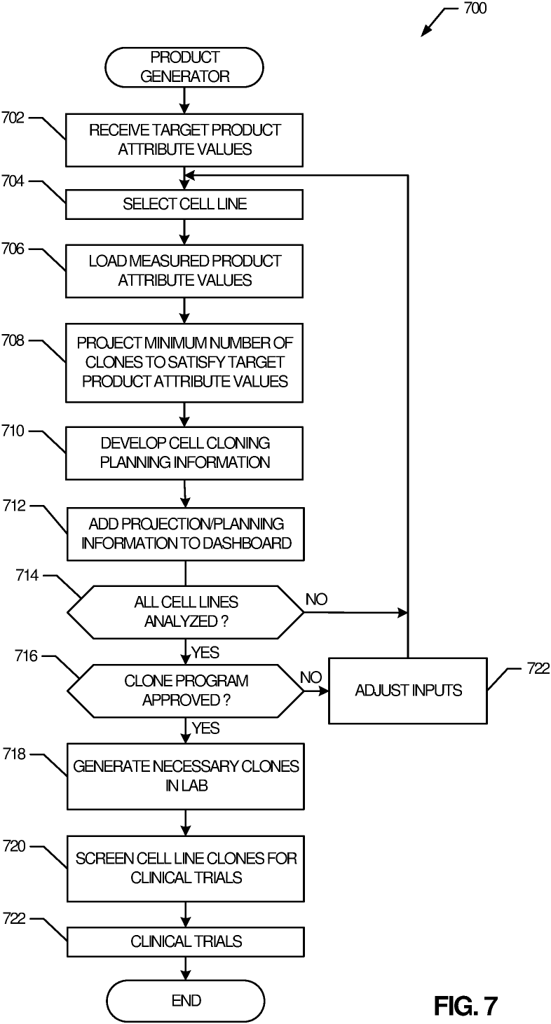

As will be described in more detail below in connection with the flowcharts of FIGS. 7 and 8, the modeling engine 206 uses the measured attributes 208 stored in the data store 214 to compute the probability of a clone within the prior, known clones 210 represented in the data store 214 satisfying conditions associated with a specified set of target product attribute values. The modeling engine 206 uses the probability to statistically project, estimate, forecast, etc. the minimum number of cell line clones that need to be generated and screened to statistically produce or result in a desired number of products having product attributes that fall within the specified set of target product attribute values. Because such projections are statistical in nature, in some examples, the projections are made for a statistical confidence level of less than one (e.g., 0.99). Further, because the set of target product attribute values do not represent all aspects of a clone that affect its clinical behavior, in some examples, the minimum number of cell line clones that needs to be generated is determined to statistically produce or result in a target number of greater than one (e.g., ten) cell line subclones that have product attributes that fall within or satisfy conditions associated with the specified set of target product attribute values.

The modeling engine 206 projects the minimum number N of clones necessary to obtain j subclones that satisfy conditions associated with the set of target product attribute values by determining a probability p that prior, known clones 210 meet the set of target product attribute values. In some examples, the probability is computed empirically based on the proportion of the prior, known clones 210 that meet the set of target product attribute values. However, to an extent the probabilities fit a known distribution, they may be computed formulaically. The modeling engine 206 computes the probabilities empirically by tabulating the number n of subclones that satisfy conditions associated with the set of target product attribute values in the set of m clones. The probability can be computed as p = n / m. The probability of exactly one of N clones satisfying conditions associated with the set of target product attribute values can be computed as p(1-p)^{N-1}. Generalizing, the probability that exactly j subclones of N clones satisfy conditions associated with the set of target product attribute values can be computed as:

$${P}_{j} = \frac{N !}{j ! \left(N - j\right) !} {p}^{j} {\left(1 - p\right)}^{N - j} . \qquad \text{EQN (1)}$$

The modeling engine 206 can thereby compute the probability P that at least k subclones satisfy conditions associated with the set of target product attribute values as:

$$P = {\sum }_{j = 0}^{k - 1} {P}_{j} = {\sum }_{j = 0}^{k - 1} \frac{N !}{j ! \left(N - j\right) !} {p}^{j} {\left(1 - p\right)}^{N - j} . \qquad \text{EQN (2)}$$

Accordingly, for a desired number of subclones k that satisfy conditions associated with a set of target product attribute values, the modeling engine 206 can solve EQN (2) to project the minimum number of clones N that need to be generated. That is the minimum number of clones N such that at least a threshold number k or a subset of size k subclones satisfies conditions associated with the target product attribute values. Because such projections are statistical in nature, in some examples, the projections are made by solving EQN (2) for P equal to a statistical confidence level C of less than one (e.g., 0.99).

When solving for k=1, P = 1 - P₀, where P₀ is the probability of finding no clone meeting the target product attribute values and is P₀=(1-p)^{N}. Accordingly, P = 1 - (1-p)^{N} can be solved for N where P is the confidence level C and p is the probability that a subclone in the set of clones satisfies conditions associated with the target product attribute values. The modeling engine 206, thus, solves for the minimum number of clones N when k=1 as N = log(1-C)/log(1-p).

For k > 1, the modeling engine 206 solves EQN (2) using numerical iteration. The modeling engine 206 starts with an initial guess for N (e.g., the number of clones in the data store 204 for the presently being considered cell line) and computes the confidence level C = P using EQN (2). The modeling engine 206 increases and decreases N until the target confidence level C (e.g., 0.99) is obtained. If the value of EQN (2) is less than the target confidence level, the modeling engine 206 increases N by, for example, one. Otherwise, the modeling engine 206 decreases N by, for example, one.

Results of the modeling engine 206 are presented in the dashboard 204, e.g., as shown in FIG. 3. In the example of FIG. 3, a table 306 is presented that shows for each of a plurality of potential host cell lines 308, the respective percentage 310 of cell line clones that are projected to fall within the specified set of target product attribute values 302 based on the percentage of previous clones of the cell line having product attribute values within the specific set of target product attribute values. For example, Cell line #3 is projected to have 95% of its clones satisfy conditions associated with the specified set of target product attribute values 302 and, thus, is a strong candidate for generating cell line clones for the scenario being investigated.

The dashboard 300 also includes an activate-able element 312 (e.g., a button) to start the modeling engine 206, a status element 314 which in FIG. 3 indicates that computations by the modeling engine 206 are complete but that might otherwise indicate computations are in progress, and another activate-able element 316 to load new, additional or different data from and/or to the data store 214 for use in current and/or future projections. While not shown in FIG. 3 for clarity of illustration, the dashboard 204, 300 may include input elements that enable the user 202 to select one or more cell lines for investigation. In some implementations, the modeling engine 206 may determine the minimum number of clones to generate at least a threshold number of subclones that satisfy conditions associated with the set of target product attribute values using empirical data from a single cell line (e.g., Cell line #3). In other implementations, the modeling engine 206 may determine the minimum number of clones using empirical data from multiple cell lines by for example, aggregating the attribute data from each cell line. In yet other implementations, the modeling engine 206 may determine the minimum number of clones using empirical data from multiple cell lines by comparing the minimum number of clones for each cell line to generate at least a threshold number of subclones that satisfy conditions associated with the set of target product attribute values and cost for generating the minimum number of clones, and identifying the cell line having the lowest minimum number, lowest cost, or any suitable combination of these. For example, the modeling engine 206 may determine that the minimum number of clones for Cell line #3 is 500 while the minimum number of clones for Cell line #1 is 400. Accordingly, the modeling engine 206 may select Cell line #1 as the cell line for generating the clones.

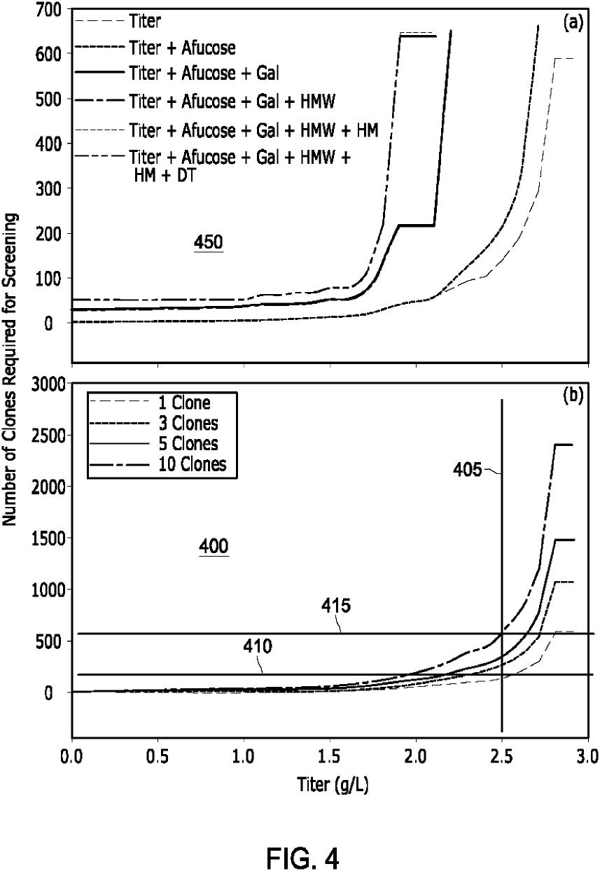

In some examples, the modeling engine 206 presents additional and/or alternative data, table, graphs, etc. that may help the user 202 understand the impact of their target product attribute values 302 on the needed number of clones. For instance, example graph 400 and/or graph 450 shown in FIG. 4 may be shown in the dashboard 204. Graphs such as graph 400 and graph 450 can be used by the user 202 to understand the impact of their target product attribute values on the number of clones that need to be generated and, thus, their impact on project costs, timelines, complexity, etc. The example graph 400 shows the projected minimum number of clones as a function of the target titer value and the desired number of subclones having titer values which meet or exceed the target titer value. For example, to identify at least ten subclones having a titer value of at least 2.5, over 500 clones need to be generated. On the other hand, to identify only at least one subclone having a titer value of at least 2.5, only about 100 clones need to be generated. Thus, nearly 400 more clones (difference between lines 410 and 415) need to be generated to find at least 10 subclones having titer values which meet or exceed the target titer value when compared to the scenario where only 1 subclone needs to have a titer value which meets or exceeds the target titer value.

In some examples, the graph 400 can be computed using Monte Carlo simulation. k random clones are extracted from the data store 204, and a maximum titer value is computed. This is repeated a number of times (e.g., one thousand) and the average of the maximum titers is computed. This is repeated for different values of k. The (k, maxavg) pairs can be plotted as shown in graph 400.

The example graph 450 shows the projected minimum number of clones as a function of the target titer value and additional target product attributes (e.g., percentage high molecular weight (%HMW), percentage afucosylation (%AFUC), percentage galactosylation (%GAL), and doubling time (DT)). As shown, as product attribute requirements are added, more clones need to be generated.

The analytical instruments 212 are configured, collectively, to obtain the physical measured attributes 208 that will be used by modeling engine 206 to make predictions, as discussed further below. Analytical instrument(s) 212 may obtain the measurements directly, and/or may obtain or facilitate indirect or “soft” sensor measurements. As used herein, the term “measurement” may refer to a value that is directly measured/sensed by an analytical instrument (e.g., one of instrument(s) 212), a value that an analytical instrument computes based upon one or more direct measurements, or a value that another device (e.g., the modeling engine 206) computes based upon one or more direct or indirect measurements. Analytical instrument(s) 212 may include instruments that are fully automated, and/or instruments that require human assistance. As just one example, analytical instrument(s) 212 may include one or more chromatograph devices (e.g., devices configured to perform size exclusion chromatography (SEC), cation exchange chromatography (CEX), and/or hydrophilic-interaction chromatography (HILIC)), one or more devices configured to obtain measurements for determining titer for a target product, one or more devices configured to directly or indirectly measure metabolite concentrations of the culture medium (e.g., glucose, glutamine, etc.), and so on.

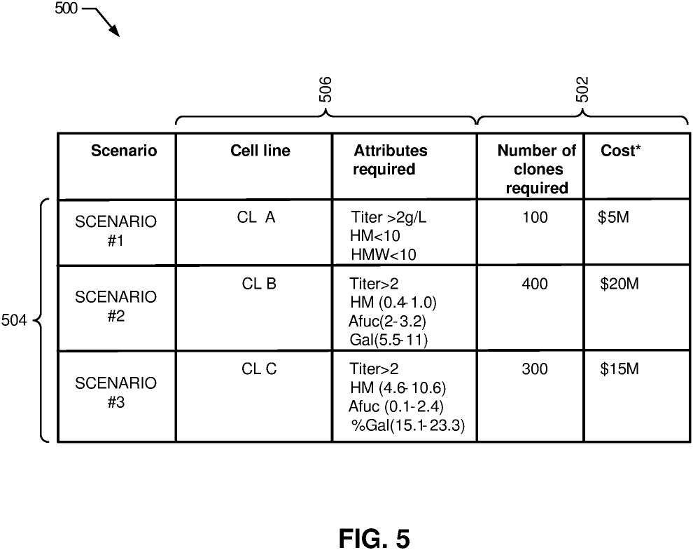

An example cell line cloning planner 216 enables the user 202 via the dashboard 204 to collect cell line cloning planning information such as an example table 500 shown in FIG. 5. The example table 500 shows cell line cloning planning information 502 (e.g., number of necessary clones and a projected cost to generate the clones) for a plurality of scenarios 506 (e.g., combinations of cell lines and target product attribute values). In some examples, the cell line cloning planner 216 is a manual tool such as a spreadsheet used by the user 202 to manually tabulate scenarios they have modeled via the dashboard 204 and modeling engine 206. In some other examples, the cell line cloning planner 216 is an automated tool that can interact with or control the modeling engine 206 to model and tabulate the results of various cell line cloning scenarios. In some examples, the cell line cloning planner 216 accesses project related information (e.g., cost to generate a clone, time to generate a clone, personnel needed, resources needed, equipment needed, etc.) in the data store 214, and uses that information to form the cell line cloning planning information 502.

The user 202, possibly in conjunction with others, uses the cell line cloning planning information 502 to determine which scenarios should be carried out. For example, which cell line clones should be generated by one or more cell line clone generators 218. Such cell line clones can be screened for further investigation in, for example, lab or clinical trials. Measured attributes 208 taken for such clones by, for example, the analytical instruments 212 can be stored in the data store 214 for use in projecting the minimum number of cell line clones to generate for future studies for other products.

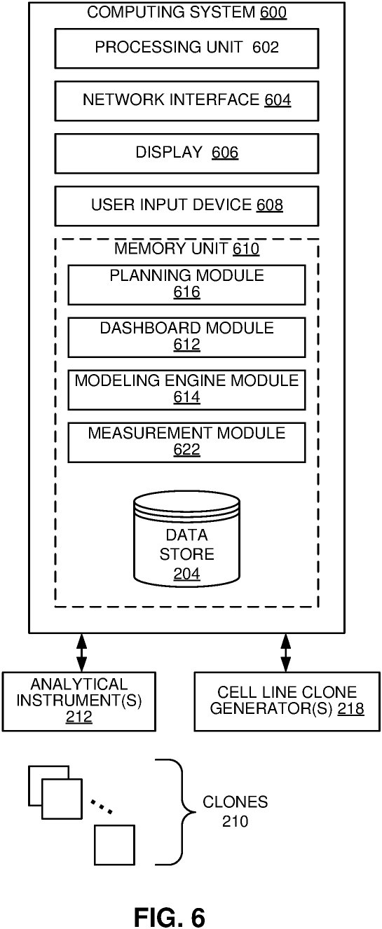

Referring now to FIG. 6, a block diagram of an example computing system 600 for determining the minimum number of cell line clones necessary to produce or result in a desired number of products having a set of target product attributes, in accordance with described embodiments is shown. The example computing system 600 may be used to, for example, implement all or part of the dashboard 204, the modeling engine 206, the data store 214 and the cell line cloning planner 216 and/or, more generally, the system 200. The computing system 600 may be a general-purpose computer that is specifically programmed to perform the operations discussed herein, or may be a special-purpose computing device.

As seen in FIG. 6, computing system 600 includes a processing unit 602, a network interface 604, a display 606, a user input device 608, and a memory unit 610. In some embodiments, the computing system 600 includes two or more computers that are either co-located or remote from each other. In these distributed embodiments, the operations described herein relating to the processing unit 602, the network interface 604 and/or the memory unit 610 may be divided among multiple processing units, network interfaces and/or memory units, respectively. The computing system 600 may be, for example, a server, a personal computer, a workstation, a self-learning machine (e.g., a neural network), or any other type of computing device.

The processing unit 602 includes one or more processors, each of which may be a programmable microprocessor that executes software or instructions stored in the memory unit 610 to execute some or all of the functions of computing system 600, as described herein. The processing unit 602 may include one or more central processing units (CPUs) and/or one or more graphics processing units (GPUs), for example. Additionally and/or alternatively, some of the processors in the processing unit 602 may be other types of processors (e.g., module-specific integrated circuits (ASICs), field-programmable gate arrays (FPGAs), digital signal processors (DSPs), etc.), and some of the functionality of the computing system 600 as described herein may instead be implemented in hardware.

The network interface 604 may include any suitable hardware (e.g., front-end transmitter and receiver hardware), firmware, and/or software configured to communicate with other computing systems and/or devices via any number and/or type(s) networks using one or more communication protocols. For example, the network interface 604 may be or include an Ethernet interface, a WiFi interface, etc.

The display 606 may use any suitable display technology (e.g., LED, OLED, LCD, etc.) to present information to a user, and the user input device 608 may be a keyboard, mouse or another suitable input device. In some embodiments, the display 606 and the user input device 608 are integrated within a single device (e.g., a touchscreen display). Generally, the display 606 and the user input device 608 may combine to enable a user to interact with graphical user interfaces (GUIs) such as the dashboard 204 discussed above with reference to FIGS. 2-5. In some embodiments, however, the computing system 600 does not include the display 606 and/or the user input device 608, or one or both of the display 606 and the user input device 608 is/are included in another computer or system (e.g., a client device) that is communicatively coupled to the computing system 600.

The memory unit 610 may include any number or type(s) of volatile or non-volatile non-transitory computer- or machine-readable storage medium, such as those disclosed above. Collectively, the memory unit 610 may store one or more software modules, the data received/used by those modules, and the data output/generated by those modules. The software modules may be embodied in software or instructions stored on one or more non-transitory computer- or machine-readable storage medium such as those disclosed above. These modules include an example dashboard module 612, an example modeling engine module 614, an example planning module 616, and an example measurement module 622. While various modules are discussed below, it is understood that those modules may be distributed among different software modules, and/or that the functionality of any one such module may be divided among two or more software modules. In some examples, the memory unit 610 implements the data store 214. Alternatively, the data store 214 is implemented separately from the computing system 600 in, for example, a server, a network drive, an external drive, etc. The data store 214 may be implemented by more than one server, network drive, external drive, etc.

A flowchart 700 representative of example processes, methods, software, computer- or machine-readable instructions, etc. for implementing the dashboard 204, the modeling engine 206, the data store 214 and the cell line cloning planner 216 and/or, more generally, the system 200. The processes, methods, software and instructions may be an executable program or portion of an executable program for execution by a processor such as the processing unit 602 of FIG. 6. The program may be embodied in software or instructions stored on a non-transitory computer- or machine-readable storage medium such as those disclosed above. Further, although the example program is described with reference to the flowchart 700 illustrated in FIG. 7, many other methods of implementing the dashboard 204, the modeling engine 206, the data store 214 and the cell line cloning planner 216 and/or, more generally, the system 200 may be implemented. For example, the order of execution of the blocks may be changed, and/or some of the blocks described may be changed, eliminated, or combined. Additionally, or alternatively, any or all of the blocks may be implemented by one or more hardware circuits (e.g., discrete and/or integrated analog and/or digital circuitry, an ASIC, a PLD, an FPGA, an FPLD, a logic circuit, etc.) structured to perform the corresponding operation without executing software or instructions.

The example program of FIG. 7 begins with the dashboard module 616. The example dashboard module 612 of FIG. 6 implements a GUI in the form of a dashboard such as the example dashboards described in connection with FIGS. 2 -5 to receive receiving a set of target product attribute values (block 702).

The dashboard module 612 receives inputs via the network interface 604 and/or the user input device 608, and provides outputs via the network interface 604 and/or the display 606. In some examples, the GUIs implemented by the dashboard module 612 are based on hypertext markup language (HTML) and displayed via a web browser executing on the computing system 600 or a computer system communicatively coupled to the computing system 600 via the network interface 604.

The modeling engine module 614 selects, or receives a selection of a cell line to consider (block 704). The modeling engine module 614 loads product attribute measurements for the cell line from the data store 204 for the clones of the selected cell line that have measurements for the target product attributes (block 706).

The modeling engine module 614 projects the minimum number N of clones necessary to obtain j subclones that satisfy conditions associated with the set of target product attribute values (block 708) by determining a probability p that clones represented in the loaded measurements meet the set of target product attribute values. In some examples, these probabilities are computed empirically. However, to an extent the probabilities fit a known distribution, they may be computed formulaically. The modeling engine module 614 computes the probabilities empirically by tabulating the number n of subclones that satisfy conditions associated with the set of target product attribute values in the set of m clones that have measurements for all of the product attributes in the set, i.e., have a measurement for each product attribute that has a target value. The probability can be computed as p = n / m. The probability of exactly one of N clones satisfying conditions associated with the set of target product attribute values can be computed as p(1-p)^{N-1}. Generalizing, the probability that exactly j subclones of N clones satisfy conditions associated with the set of target product attribute values can be computed using EQN (1) shown above.

The modeling engine module 614 can thereby compute the probability P that at least k subclones satisfy conditions associated with the set of target product attribute values using EQN (2) shown above.

Accordingly, for a desired number of subclones k that satisfy conditions associated with a set of target product attribute values, the modeling engine module 614 can solve EQN (2) to project the minimum number of clones N that need to be generated. That is the minimum number of clones N such that a threshold number k or subset of size k satisfy conditions associated with the target product attribute values. Because such projections are statistical in nature, in some examples, the projections are made by solving EQN (2) for P equal to a statistical confidence level C of less than one (e.g., 0.99).

When solving for k=1, P = 1 - P₀, where P₀ is the probability of finding no clone meeting the target product attribute values and is P₀=(1-p)^{N}. Accordingly, P = 1 - (1-p)^{N} can be solved for N where P is the confidence level C, and p is the probability that a subclone in the set of clones satisfies conditions associated with the target product attribute values. The modeling engine module 614, thus, solves for the minimum number of clones N when k=1 as N = log(1-C)/log(1-p).

For k > 1, the modeling engine module 614 solves EQN (2) using numerical iteration. The modeling engine module 614 starts with an initial guess for N (e.g., the number of clones in the data store 204 for the presently being considered cell line) and computes the confidence level C = P using EQN (2). The modeling engine module 614 increases and decreases N until the target confidence level C (e.g., 0.99) is obtained. If the value of EQN (2) is less than the target confidence level, the modeling engine module 614 increases N by, for example, one. Otherwise, the modeling engine module 614 decreases N by, for example, one.

The example planning module 616 enables the user 202 to collect cell line cloning planning information such as in example table 500 shown in FIG. 5. (block 710) In some examples, the planning module 616 is a manual tool such as a spreadsheet used by the user 202 to manually tabulate scenarios they have modeled via the dashboard module 612 and/or the modeling engine module 614. In some other examples, the planning module 616 is an automated tool that can interact with or control the modeling engine module 614 to model and tabulate the results of various cell line cloning scenarios. In some examples, the planning module 616 accesses project related information (e.g., cost to generate a clone, time to generate a clone, personnel needed, resources needed, equipment needed, etc.) in the data store 214, and uses that information to form the cell line cloning planning information 502. In some examples, the planning module 616 implements an interface based on HTML and displayed via a web browser executing on the computing system 600 or a computer system communicatively coupled to the computing system 600 via the network interface 604.

After the modeling engine module 614 makes minimum projections for each of the cell lines (block 714), a user can review the cloning planning information collected by the planning module and approve a cloning program (block 716). If the cloning program is approved (block 716), the minimum number of clones can be generated (block 718) and screened (e.g., by measuring a set of resultant product attribute values for each clone) (block 720). Clones that pass screening (e.g., based on a comparison of the resultant product attribute values to the target product attribute values) can be studied further in laboratory or clinical trials (block 722). The example program of FIG. 7 then ends.

Returning to block 716, if the cloning program is not approved, the user can adjust cell line selections and/or target product attribute values (block 722), and the modeling engine module 614 can update projections for the minimum number of clones needed to satisfy conditions associated with the target product attribute values.

Returning to block 714, if not all selected cell lines have been considered, flow returns to block 704 to consider a next cell line.

The example measurement module 622 collects values of various attributes associated with cell line clones. For example, the measurement module 622 may receive measurements directly from analytical instrument(s) 212. Additionally or alternatively, the measurement module 622 may receive information stored in a measurement database (not shown) and/or information entered by a user (e.g., via the user input device 608).

While the embodiments described herein can be used, inter alia, to minimize the number of clones necessary to generate a biosimilar having a desired set of product attributes, one of skill in the art will recognize that the disclosure herein has other uses outside of biosimilar development and it is to be understood that the invention is not intended to be limited to biosimilar development.

In some cases it may be necessary to generate a cell line capable of producing a protein of interest in a highly similar fashion such as by minimizing the differences to the primary amino acid sequence of the produced protein as compared to the protein of interest, ensuring similar modifications are made to the produced protein as compared to the protein of interest (e.g. similar glycosylation patterns), and reproducibly producing the protein displaying a similar higher order structure as the protein of interest (e.g. protein folding). As many cell culture conditions including the choice of cell line can influence these modifications, in some cases there is a need to screen numerous cell lines and clones for their ability to produce a highly similar reference protein. The disclosure herein provides an improvement in this respect by reducing the number of clones that need to be generated for potential screening.

Example methods and systems for determining a minimum number of cell line clones necessary to produce a product having a set of target product attributes are disclosed. Further examples and combinations thereof include at least the following.

Example 1 is a method including generating at least one cell line capable of expressing a polypeptide; measuring, using one or more analytical instruments, a plurality of measured product attribute values of a plurality of clones of a candidate cell line; receiving inputs, via a user interface, representing a set of target product attribute values for a product; projecting, by one or more processors based upon the plurality of measured values, a minimum number of subject clones of the product using the candidate cell line necessary to produce a subset of the subject clones having product attributes that satisfy one or more conditions associated with the set of target values; and generating the projected minimum number of subject clones of the product using the candidate cell line.

Example 2 is the method of example 1, wherein the subset of the subject clones represents a threshold number of the clones having product attributes that satisfy one or more conditions associated with the set of target values.

Example 3 is the method of example 1 or example 2, wherein the projecting includes: computing a probability that one of the plurality of clones satisfies one or more conditions associated with the set of target values based upon a total number of the plurality of clones and a number of the plurality of clones having product attributes that satisfy the one or more conditions associated with set of target product attribute values; and projecting the minimum number of subject clones based upon the probability.

Example 4 is the method of example 3, wherein the probability is a first probability, and wherein the projecting further includes: receiving, via a user interface, a confidence level value indicative of a second probability in which the subset of the subject clones results in at least a threshold number of clones having product attributes that satisfy the one or more conditions associated with the target values; and projecting the minimum number of subject clones as a function of the confidence level value, the first probability, and the threshold number of clones.

Example 5 is the method of example 4, wherein projecting the minimum number of subject clones includes solving for the minimum number N of subject clones given the threshold number k of clones satisfying the one or more conditions associated with the set of target product attribute values and the confidence level C is:

$$C = {\sum }_{j = 0}^{k - 1} \frac{N !}{j ! \left(N - j\right) !} {p}^{j} {\left(1 - p\right)}^{N - j} .$$

Example 6 is the method of example 4 or example 5, wherein the threshold number of clones is one, the minimum number of subject clones (n) is determined as:

$$n = \frac{log \left(1 - C\right)}{log \left(1 - p\right)} ,$$

C is the confidence level value, and p is the first probability.

Example 7 is the method of any of examples 3 to 6, wherein the probability is an empirical probability.

Example 8 is the method of any of examples 1 to 7, wherein the plurality of measured values includes at least one of a titer, a percentage high molecular weight, a percentage high mannose, a percentage Afucosylation, a percentage Galactosylation, or a doubling time.

Example 9 is the method of any of examples 1 to 8, wherein the candidate cell line is a first candidate cell line, the minimum number of the subject clones is a first minimum number, and further comprising: measuring, using the one or more analytical instruments, another plurality of measured product attribute values of another plurality of clones of a second candidate cell line; projecting, by the one or more processors based upon the another plurality of measured values, a second minimum number of other subject clones of the product using the second candidate cell line necessary to produce a subset of the other subject clones having product attributes that satisfy the one or more conditions associated with the set of target values; and selecting between generating the subject clones using the first candidate cell line and generating the other subject clones using the second candidate cell line based upon at least one of the first minimum number, the second minimum number, a first cost to generate a first clone based upon the first candidate cell line, and a second cost to generate a second clone based upon the second candidate cell line.

Example 10 is the method of any of examples 1 to 9, further comprising: measuring, using the one or more analytical instruments, a set of resultant product attribute values for each of the subject clones; and identifying one or more of the subject clones for additional testing based upon comparisons of the sets of measured resultant values and the set of target values.

Example 11 is the method of any of examples 1 to 10, further comprising: projecting, by the one or more processors for each of a plurality of sets of target values, a minimum number of subject clones of the product to produce to generate at least a subset of clones having product attributes that satisfy the one or more conditions associated with the set of target values; and displaying, by the one or more processors, a graph or chart of the minimum numbers of subject clones as a function of the plurality of sets of target values.

Example 12 is a non-transitory, computer-readable medium storing instructions that, when executed by a processor, cause a computing system to: access a plurality of measured product attribute values of a plurality of clones of a candidate cell line; receive inputs, via a user interface, representing a set of target product attribute values for a product; project, by one or more processors based upon the plurality of measured values, a minimum number of subject clones of the product using the candidate cell line necessary to produce a subset of the subject clones having product attributes that satisfy one or more conditions associated with the set of target values; and generate the projected minimum number of subject clones of the product using the candidate cell line.

Example 13 is the non-transitory, computer-readable medium of example 12, wherein the instructions, when executed by the processor, cause the computing system to: compute a probability that one of the plurality of clones satisfies the one or more conditions associated with the set of target values based upon a total number of the plurality of clones and a number of the plurality of clones having product attributes that satisfy the one or more conditions associated with the set of target product attribute values; and project the minimum number of subject clones based upon the probability.

Example 14 is the non-transitory, computer-readable medium of example 13, wherein the instructions, when executed by the processor, cause the computing system to: compute a probability that one of the plurality of clones satisfies the one or more conditions associated with the set of target values based upon a total number of the plurality of clones and a number of the plurality of clones having product attributes that satisfy the one or more conditions associated with the set of target product attribute values; and project the minimum number of subject clones based upon the probability.

Example 15 is the non-transitory, computer-readable medium of example 14, wherein the probability is a first probability, and wherein the instructions, when executed by the processor, cause the computing system to: receive, via a user interface, a confidence level value indicative of a second probability in which the subset of the subject clones results in at least a threshold number of clones having product attributes that satisfy the one or more conditions associated with the target values; and project the minimum number of subject clones as a function of the confidence level value, the first probability, and the threshold number of clones.

Example 16 is the non-transitory, computer-readable medium of example 15, wherein the instructions, when executed by the processor, cause the computing system to project the minimum number of subject clones by solving for the minimum number N of subject clones given the threshold number k of clones satisfying the one or more conditions associated with the set of target product attribute values and the confidence level C is:

$$C = {\sum }_{j = 0}^{k - 1} \frac{N !}{j ! \left(N - j\right) !} {p}^{j} {\left(1 - p\right)}^{N - j} .$$

Example 17 is the non-transitory, computer-readable medium of example 15 or example 16, wherein the threshold number of clones is one, the minimum number of subject clones (n) is determined as:

$$n = \frac{log \left(1 - C\right)}{log \left(1 - p\right)} ,$$

C is the confidence level value, and p is the first probability.

Example 18 is the non-transitory, computer-readable medium of any of examples 12 to 17, wherein the candidate cell line is a first candidate cell line, the minimum number of the subject clones is a first minimum number, and wherein the instructions, when executed by the processor, cause the computing system to: measure, using the one or more analytical instruments, another plurality of measured product attribute values of another plurality of clones of a second candidate cell line; project, by the one or more processors based upon the another plurality of measured values, a second minimum number of other subject clones of the product using the second candidate cell line necessary to produce a subset of the other subject clones having product attributes that satisfy the one or more conditions associated with the set of target values; and select between generating the subject clones using the first candidate cell line and generating the other subject clones using the second candidate cell line based upon at least one of the first minimum number, the second minimum number, a first cost to generate a first clone based upon the first candidate cell line, and a second cost to generate a second clone based upon the second candidate cell line.

Example 19 is the non-transitory, computer-readable medium of examples 12 to 18, further comprising: measuring, using the one or more analytical instruments, a set of resultant product attribute values for each of the subject clones; and identifying one or more of the subject clones for additional testing based upon comparisons of the sets of measured resultant values and the set of target values.

Example 20 is a system to produce a minimum number of cell line clones necessary to produce a product having a set of target product attributes, the system comprising: analytical instruments configured to measure a plurality of measured product attribute values of a plurality of clones of a candidate cell line; a user interface configured to receive inputs representing a set of target product attribute values for a product; a modeling engine configured to project, based upon the plurality of measured values, a minimum number of subject clones of the product using the candidate cell line necessary to produce a subset of the subject clones having product attributes that satisfy one or more conditions associated with the set of target values; and a cell line clone generator configured to generate the projected minimum number of subject clones of the product using the candidate cell line.

Example 21 is the system of example 20, wherein the modeling engine is configured to project the minimum number by: determining a probability that one of the plurality of clones satisfies the one or more conditions associated with the set of target values based upon a total number of the plurality of clones and a number of the plurality of clones having product attributes that satisfy the one or more conditions associated with the set of target product attribute values; and projecting the minimum number of subject clones based upon the probability.

Example 22 is the system of example 21, wherein the subset of the subject clones represents a threshold number of the subject clones having product attributes that satisfy the one or more conditions associated with the set of target values.

Example 23 is the system of any of example 22, the modeling engine is configured to project the minimum number by: computing a probability that one of the plurality of clones satisfies the one or more conditions associated with the set of target values based upon a total number of the plurality of clones and a number of the plurality of clones having product attributes that satisfy the one or more conditions associated with the set of target product attribute values; and projecting the minimum number of subject clones based upon the probability.

Example 24 is the system of example 23, wherein the probability is a first probability, and wherein the modeling engine is further configured to: receiving, via a user interface, a confidence level value indicative of a second probability in which the subset of the subject clones results in at least a threshold number of clones having product attributes that satisfy the one or more conditions associated with the target values; and projecting the minimum number of subject clones as a function of the confidence level value, the first probability, and the threshold number of clones.

Example 25 is the system of example 24, wherein the modeling engine is further configured to project the minimum number by solving for the minimum number N of subject clones given the threshold number k of clones satisfying the one or more conditions associated with the set of target product attribute values and the confidence level C is

$$C = {\sum }_{j = 0}^{k - 1} \frac{N !}{j ! \left(N - j\right) !} {p}^{j} {\left(1 - p\right)}^{N - j} .$$

Example 26 is the system of example 24, wherein the threshold number of clones is one, the minimum number of subject clones (n) is determined as:

$$n = \frac{log \left(1 - C\right)}{log \left(1 - p\right)} ,$$

C is the confidence level value, and p is the first probability.

Example 27 is the system of any of examples 20 to 26, wherein the candidate cell line is a first candidate cell line, the minimum number of the subject clones is a first minimum number, and further comprising: measuring, using the one or more analytical instruments, another plurality of measured product attribute values of another plurality of clones of a second candidate cell line; projecting, by the one or more processors based upon the another plurality of measured values, a second minimum number of other subject clones of the product using the second candidate cell line necessary to produce a subset of the other subject clones having product attributes that satisfy the one or more conditions associated with the set of target values; and selecting between generating the subject clones using the first candidate cell line and generating the other subject clones using the second candidate cell line based upon at least one of the first minimum number, the second minimum number, a first cost to generate a first clone based upon the first candidate cell line, and a second cost to generate a second clone based upon the second candidate cell line.

Example 28 is the system of any of examples 20 to 27, further comprising: measuring, using the one or more analytical instruments, a set of resultant product attribute values for each of the subject clones; and identifying one or more of the subject clones for additional testing based upon comparisons of the sets of measured resultant values and the set of target values.

Use of “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise. A device or structure that is “configured” in a certain way is configured in at least that way, but may also be configured in ways that are not listed.

Further, as used herein, the expressions “in communication,” “coupled” and “connected,” including variations thereof, encompasses direct communication and/or indirect communication through one or more intermediary components, and does not require direct mechanical or physical (e.g., wired) communication and/or constant communication, but rather additionally includes selective communication at periodic intervals, scheduled intervals, aperiodic intervals, and/or one-time events. The embodiments are not limited in this context.

Further still, unless expressly stated to the contrary, “or” refers to an inclusive or and not to an exclusive or. For example, “A, B or C” refers to any combination or subset of A, B, C such as (1) A alone, (2) B alone, (3) C alone, (4) A with B, (5) A with C, (6) B with C, and (7) A with B and with C. As used herein, the phrase “at least one of A and B” is intended to refer to any combination or subset of A and B such as (1) at least one A, (2) at least one B, and (3) at least one A and at least one B. Similarly, the phrase “at least one of A or B” is intended to refer to any combination or subset of A and B such as (1) at least one A, (2) at least one B, and (3) at least one A and at least one B.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

Although the systems, methods, devices, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. A method for determining a minimum number of cell line clones necessary to produce a product having a set of target product attributes, the method comprising:
generating at least one cell line capable of expressing a polypeptide;
measuring, using one or more analytical instruments, a plurality of measured product attribute values of a plurality of clones of a candidate cell line;
receiving inputs, via a user interface, representing a set of target product attribute values for a product;
projecting, by one or more processors based upon the plurality of measured values, a minimum number of subject clones of the product using the candidate cell line necessary to produce a subset of the subject clones having product attributes that satisfy one or more conditions associated with the set of target values; and
generating the projected minimum number of subject clones of the product using the candidate cell line.

2. The method of claim 1, wherein the subset of the subject clones represents a threshold number of the subject clones having product attributes that satisfy the one or more conditions associated with the set of target values.

3. The method of claim 1, wherein the projecting includes:
computing a probability that one of the plurality of clones satisfies the one or more conditions associated with the set of target values based upon a total number of the plurality of clones and a number of the plurality of clones having product attributes that satisfy the one or more conditions associated with the set of target product attribute values; and
projecting the minimum number of subject clones based upon the probability.

4. The method of claim 3, wherein the probability is a first probability, and wherein the projecting further includes:
receiving, via a user interface, a confidence level value indicative of a second probability in which the subset of the subject clones results in at least a threshold number of clones having product attributes that satisfy the one or more conditions associated with the target values; and
projecting the minimum number of subject clones as a function of the confidence level value, the first probability, and the threshold number of clones.

5. The method of claim 4, wherein projecting the minimum number of subject clones includes solving for the minimum number N of subject clones given the threshold number k of clones satisfying the one or more conditions associated with the set of target product attribute values and the confidence level C is:
$$C = {\sum }_{j = 0}^{k - 1} \frac{N !}{j ! \left(N - j\right) !} {p}^{j} {\left(1 - p\right)}^{N - j}$$
wherein p is the first probability.

6. The method of claim 4, wherein
the threshold number of clones is one,
the minimum number of subject clones (n) is determined as:
$$n = \frac{log \left(1 - C\right)}{log \left(1 - p\right)} ,$$
C is the confidence level value, and
p is the first probability.

7. The method of claim 3, wherein the probability is an empirical probability.

8. The method of claim 1, wherein the plurality of measured values includes at least one of a titer, a percentage high molecular weight, a percentage high mannose, a percentage Afucosylation, a percentage Galactosylation, or a doubling time.

9. The method of claim 1, wherein the candidate cell line is a first candidate cell line, the minimum number of the subject clones is a first minimum number, and further comprising:
measuring, using the one or more analytical instruments, another plurality of measured product attribute values of another plurality of clones of a second candidate cell line;
projecting, by the one or more processors based upon the another plurality of measured values, a second minimum number of other subject clones of the product using the second candidate cell line necessary to produce a subset of the other subject clones having product attributes that satisfy the one or more conditions associated with the set of target values; and
selecting between generating the subject clones using the first candidate cell line and generating the other subject clones using the second candidate cell line based upon at least one of the first minimum number, the second minimum number, a first cost to generate a first clone based upon the first candidate cell line, and a second cost to generate a second clone based upon the second candidate cell line.

10. The method of claim 1, further comprising:
measuring, using the one or more analytical instruments, a set of resultant product attribute values for each of the subject clones; and
identifying one or more of the subject clones for additional testing based upon comparisons of the sets of measured resultant values and the set of target values.

11. The method of claim 1, further comprising:
projecting, by the one or more processors for each of a plurality of sets of target values, a minimum number of subject clones of the product to produce to generate at least a subset of clones having product attributes that satisfy the one or more conditions associated with the set of target values; and
displaying, by the one or more processors, a graph or chart of the minimum numbers of subject clones as a function of the plurality of sets of target values.

12. A non-transitory, computer-readable medium storing instructions that, when executed by a processor, cause a computing system to:
access a plurality of measured product attribute values of a plurality of clones of a candidate cell line;
receive inputs, via a user interface, representing a set of target product attribute values for a product;
project, by one or more processors based upon the plurality of measured values, a minimum number of subject clones of the product using the candidate cell line necessary to produce a subset of the subject clones having product attributes that satisfy one or more conditions associated with the set of target values; and
generate the projected minimum number of subject clones of the product using the candidate cell line.

13. The non-transitory, computer-readable medium of claim 12, wherein the instructions, when executed by the processor, cause the computing system to:
compute a probability that one of the plurality of clones satisfies the one or more conditions associated with the set of target values based upon a total number of the plurality of clones and a number of the plurality of clones having product attributes that satisfy the one or more conditions associated with the set of target product attribute values; and
project the minimum number of subject clones based upon the probability.

14. The non-transitory, computer-readable medium of claim 13, wherein the instructions, when executed by the processor, cause the computing system to:
compute a probability that one of the plurality of clones satisfies the one or more conditions associated with the set of target values based upon a total number of the plurality of clones and a number of the plurality of clones having product attributes that satisfy the one or more conditions associated with the set of target product attribute values; and
project the minimum number of subject clones based upon the probability.

15. The non-transitory, computer-readable medium of claim 14, wherein the probability is a first probability, and wherein the instructions, when executed by the processor, cause the computing system to:
receive, via a user interface, a confidence level value indicative of a second probability in which the subset of the subject clones results in at least a threshold number of clones having product attributes that satisfy the one or more conditions associated with the target values; and
project the minimum number of subject clones as a function of the confidence level value, the first probability, and the threshold number of clones.

16. The non-transitory, computer-readable medium of claim 15, wherein the instructions, when executed by the processor, cause the computing system to project the minimum number of subject clones by solving for the minimum number N of subject clones given the threshold number k of clones satisfying the one or more conditions associated with the set of target product attribute values and the confidence level C is:
$$C = {\sum }_{j = 0}^{k - 1} \frac{N !}{j ! \left(N - j\right) !} {p}^{j} {\left(1 - p\right)}^{N - j}$$
wherein p is the first probability.

17. The non-transitory, computer-readable medium of claim 15, wherein
the threshold number of clones is one,
the minimum number of subject clones (n) is determined as:
$$n = \frac{log \left(1 - C\right)}{log \left(1 - p\right)} ,$$
C is the confidence level value, and
p is the first probability.

18. The non-transitory, computer-readable medium of claim 12, wherein the candidate cell line is a first candidate cell line, the minimum number of the subject clones is a first minimum number, and wherein the instructions, when executed by the processor, cause the computing system to:
measure, using the one or more analytical instruments, another plurality of measured product attribute values of another plurality of clones of a second candidate cell line;
project, by the one or more processors based upon the another plurality of measured values, a second minimum number of other subject clones of the product using the second candidate cell line necessary to produce a subset of the other subject clones having product attributes that satisfy the one or more conditions associated with the set of target values; and
select between generating the subject clones using the first candidate cell line and generating the other subject clones using the second candidate cell line based upon at least one of the first minimum number, the second minimum number, a first cost to generate a first clone based upon the first candidate cell line, and a second cost to generate a second clone based upon the second candidate cell line.

19. The non-transitory, computer-readable medium of claim 12, further comprising:
measuring, using the one or more analytical instruments, a set of resultant product attribute values for each of the subject clones; and
identifying one or more of the subject clones for additional testing based upon comparisons of the sets of measured resultant values and the set of target values.

20. A system to produce a minimum number of cell line clones necessary to produce a product having a set of target product attributes, the system comprising:
analytical instruments configured to measure a plurality of measured product attribute values of a plurality of clones of a candidate cell line;
a user interface configured to receive inputs representing a set of target product attribute values for a product;
a modeling engine configured to project, based upon the plurality of measured values, a minimum number of subject clones of the product using the candidate cell line necessary to produce a subset of the subject clones having product attributes that satisfy one or more conditions associated with the set of target values; and
a cell line clone generator configured to generate the projected minimum number of subject clones of the product using the candidate cell line.

21. The system of claim 20, wherein the modeling engine is configured to project the minimum number by:
determining a probability that one of the plurality of clones satisfies the one or more conditions associated with the set of target values based upon a total number of the plurality of clones and a number of the plurality of clones having product attributes that satisfy the one or more conditions associated with the set of target product attribute values; and
projecting the minimum number of subject clones based upon the probability.

22. The system of claim 20, wherein the subset of the subject clones represents a threshold number of the subject clones having product attributes that satisfy the one or more conditions associated with the set of target values.

23. The system of claim 20, the modeling engine is configured to project the minimum number by:
computing a probability that one of the plurality of clones satisfies the one or more conditions associated with the set of target values based upon a total number of the plurality of clones and a number of the plurality of clones having product attributes that satisfy the one or more conditions associated with the set of target product attribute values; and
projecting the minimum number of subject clones based upon the probability.

24. The system of claim 23, wherein the probability is a first probability, and wherein the modeling engine is further configured to:
receiving, via a user interface, a confidence level value indicative of a second probability in which the subset of the subject clones results in at least a threshold number of clones having product attributes that satisfy the one or more conditions associated with the target values; and
projecting the minimum number of subject clones as a function of the confidence level value, the first probability, and the threshold number of clones.

25. The system of claim 24, wherein the modeling engine is further configured to project the minimum number by solving for the minimum number N of subject clones given the threshold number k of clones satisfying the one or more conditions associated with the set of target product attribute values and the confidence level C is:
$$C = {\sum }_{j = 0}^{k - 1} \frac{N !}{j ! \left(N - j\right) !} {p}^{j} {\left(1 - p\right)}^{N - j}$$
wherein p is the first probability.

26. The system of claim 24, wherein
the threshold number of clones is one,
the minimum number of subject clones (n) is determined as:
$$n = \frac{log \left(1 - C\right)}{log \left(1 - p\right)} ,$$
C is the confidence level value, and
p is the first probability.

27. The system of claim 20, wherein the candidate cell line is a first candidate cell line, the minimum number of the subject clones is a first minimum number, and further comprising:
measuring, using the one or more analytical instruments, another plurality of measured product attribute values of another plurality of clones of a second candidate cell line;
projecting, by the one or more processors based upon the another plurality of measured values, a second minimum number of other subject clones of the product using the second candidate cell line necessary to produce a subset of the other subject clones having product attributes that satisfy the one or more conditions associated with the set of target values; and
selecting between generating the subject clones using the first candidate cell line and generating the other subject clones using the second candidate cell line based upon at least one of the first minimum number, the second minimum number, a first cost to generate a first clone based upon the first candidate cell line, and a second cost to generate a second clone based upon the second candidate cell line.

28. The system of claim 20, further comprising:
measuring, using the one or more analytical instruments, a set of resultant product attribute values for each of the subject clones; and
identifying one or more of the subject clones for additional testing based upon comparisons of the sets of measured resultant values and the set of target values.
